(19) United States
(12) Patent Application Publication
(10) Pub. No.: US 2011/0313359 A1
(43) Pub. Date: Dec. 22, 2011

(54) RETENTION DEVICE FOR GASTROSTOMY TUBE AND LOW PROFILE GASTROSTOMY DEVICE

(76) Inventor: Stanley A. Cohen, Atlanta, GA (US)

(21) Appl. No.: 13/164,040

(22) Filed: Jun. 20, 2011

Related U.S. Application Data
(60) Provisional application No. 61/356,694, filed on Jun. 21, 2010.

(51) Int. Cl. A61M 39/00

(52) U.S. Cl. 604/175

(57) ABSTRACT

A gastrostomy tube including a retention device includes an elongated hollow shaft having a proximal first end and a distal second end and at least one retention member extending from the distal second end of the shaft. The at least one retention member moves between a retracted orientation and an open orientation.

### CROSS REFERENCE TO RELATED APPLICATION

This application claims the benefit of U.S. Provisional Patent Application Ser. No. 61/356,694, entitled “RETENTION DEVICE FOR GASTROSTOMY TUBE”, filed Jun. 21, 2010.

### BACKGROUND OF THE INVENTION

1. Field of the Invention

The present disclosure relates to a gastrostomy tube and, in particular, an improved retention device for a gastrostomy tube and a low profile gastrostomy device.

2. Description of the Related Art

Gastrostomy tubes are used to deliver nutrients and medications enterally to individuals who for any number of reasons are unable to eat or swallow. These tubes are usually changed into low profile devices, often referred to as buttons since their exteriors are close to the skin and opened (often like a snap).

A conventional gastrostomy feeding tube includes a retention device using an inflatable balloon to retain the gastrostomy feeding tube in place. An example of such a conventional gastrostomy feeding tube and retention device is the MIC-KEY low profile gastrostomy feeding tube offered by Kimberly Clark.

To use the conventional gastrostomy feeding tube, a passage (stoma) is formed through the skin and gastric wall of the stomach. The retention device and feeding tube are then inserted through the passage while the balloon is in an uninflated position. The balloon portion of the retention device is inserted through the passage into the stomach. Once in the stomach, the balloon is inflated to retain the gastrostomy feeding tube in place.

Gastrostomy feeding tube devices employing inflatable balloons have the current advantage because they are easily inserted and inflated so that the internal balloon holds them in place. However, patients who are unconscious or unruly can remove them relatively easily. Also the balloons can wear out within a few months. More rigid mushroom type retention devices are also currently on the market but these are difficult to insert and often traumatic to remove.

There is a need, therefore, for an improved retention device for a gastrostomy tube.

### SUMMARY OF THE INVENTION

It is, therefore, an object of the present invention to provide a gastrostomy tube including a retention device. The gastrostomy tube includes an elongated hollow shaft having a proximal first end and a distal second end and at least one retention member extending from the distal second end of the shaft. The at least one retention member moves between a retracted orientation and an open orientation.

It is also an object of the present invention to provide a gastrostomy tube wherein the at least one retention member is a spline resiliently biased in a splayed configuration.

It is another object of the present invention to provide a gastrostomy tube wherein the spline is composed of a shape memory alloy.

It is a further object of the present invention to provide a gastrostomy tube wherein the at least one retention member includes a plurality of splines resiliently biased in a splayed configuration.

It is also an object of the present invention to provide a gastrostomy tube including a concentric sleeve wrapped about the shaft for sliding longitudinally along the shaft.

It is another object of the present invention to provide a gastrostomy tube wherein the at least one retention member is a spline resiliently biased in a splayed configuration.

It is a further object of the present invention to provide a gastrostomy tube wherein movement of the sleeve relative to the shaft causes the at least one retention member to move between a retracted orientation and an open orientation.

It is also an object of the present invention to provide a gastrostomy tube wherein a lower edge of the sleeve contacts a proximal end of the at least one retention member causing the at least one retention member to move to a retracted orientation.

It is another object of the present invention to provide a gastrostomy tube wherein the spline is composed of a shape memory alloy.

It is a further object of the present invention to provide a gastrostomy tube including a locking mechanism for selectively locking the shaft relative to the sleeve.

It is also an object of the present invention to provide a gastrostomy tube including a button for closing the hollow shaft.

It is another object of the present invention to provide a gastrostomy tube wherein the at least one retention member is a mesh device biased to an expanded form.

It is a further object of the present invention to provide a gastrostomy tube wherein the mesh device is a shape memory metal alloy.

It is also an object of the present invention to provide a gastrostomy tube wherein the mesh device is a plastic material.

It is another object of the present invention to provide a gastrostomy tube including an introducer used in controlling the orientation of the mesh device.

It is a further object of the present invention to provide a gastrostomy tube wherein the introducer includes a shaft having a distal end shaped and dimensioned to engage the mesh device.

It is also an object of the present invention to provide a wherein the distal end of the shaft includes a point tip.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIGS. 1A and 1B illustrate one embodiment of a retention device for a gastrostomy tube of the present disclosure. FIG. 1C illustrates a removal device for use in conjunction with the retention device disclosed in FIGS. 1A and 1B.

FIGS. 2A and 2B illustrate an alternate connection mechanism for securing a retention shaft to the outer sleeve.

FIGS. 3A-C illustrate another embodiment of a retention device for a gastrostomy tube of the present disclosure.

FIGS. 4A-C illustrate yet another embodiment of a retention device for a gastrostomy tube of the present disclosure.

FIG. 5 illustrates an alternate introducer for use in conjunction with the retention device disclosed in FIGS. 4A-4C.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

In accordance with the present invention, a gastrostomy feeding tube, in particular, a retention device of the gastrostomy tube, is disclosed. The gastrostomy tube includes an elongated hollow shaft having a proximal first end and a distal second end with at least one retention member extending from the distal second end of the shaft. The at least one retention member moves between a retracted orientation and an open orientation.

The improved retention device for a gastrostomy feeding tube of the present disclosure is designed, in particular, to meet and overcome the aforementioned disadvantages of conventional retention devices for gastrostomy feeding tubes. In an exemplary embodiment, the retention device for a gastrostomy tube of the present disclosure includes an umbrella-like device having a plurality of fingers or splines that may be exposed allowing the fingers or splines at one (distal) end of the retention member to splay outwardly of each other to retain the device in place or alternatively withdraw the fingers or splines to allow the device to either be inserted through a body passageway formed through the skin and stomach wall or alternatively withdrawn from the body passageway.

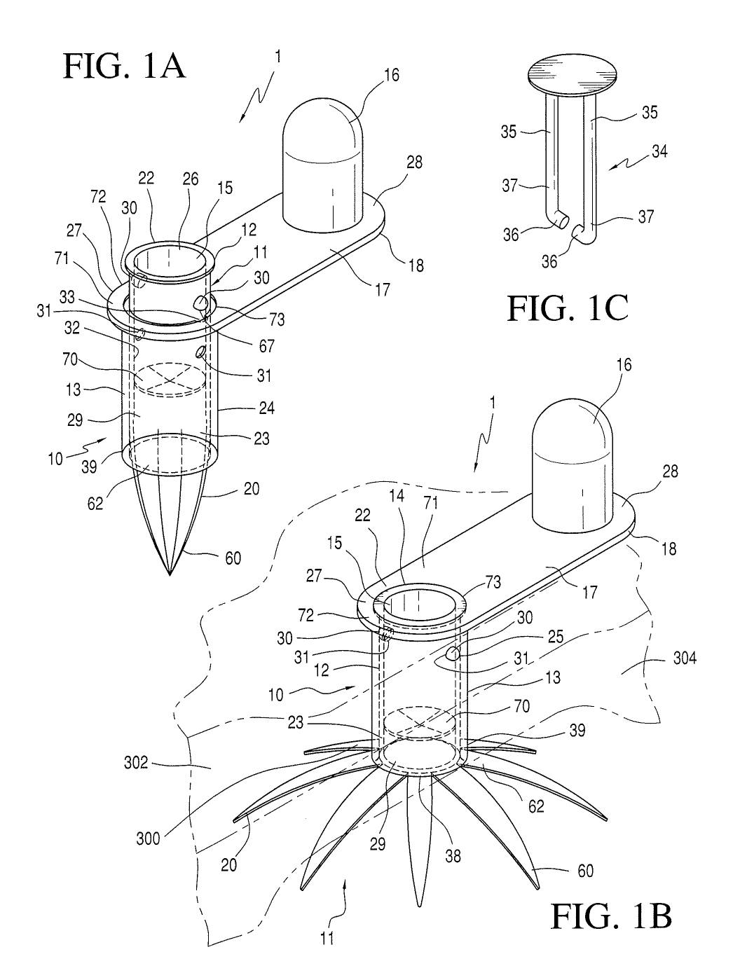

Referring more specifically to the drawings in which like reference numerals refer to like elements throughout the several views, exemplary non-limiting embodiments of a retention member for a gastrostomy tube of the present disclosure is illustrated. Referring to FIGS. 1A and 1B, one embodiment of a retention device 10 for a gastrostomy tube 1 of the present disclosure is illustrated. FIGS. 1A and 1B illustrate a gastrostomy tube 1 including a retention device 10. The retention device 10, and ultimately the gastrostomy tube 1, is composed of a retention shaft assembly 11 and a concentric outer sleeve 13 positioned about the retention shaft assembly 11 for movement relative thereto. The retention shaft assembly 11 includes a hollow retention shaft 12 with retention members 20 extending therefrom.

As will be appreciated based upon the following disclosure, the concentric outer sleeve 13 includes a cylindrical body 24 in which the retention shaft assembly 11 is positioned for movement therein. As such, the cylindrical body 24 of the concentric outer sleeve 13 includes a central passageway 29 in which the retention shaft assembly 11 is positioned for movement between a retracted position/orientation (see FIG. 1A) and an open position/orientation (see FIG. 1B).

The retention shaft 12 is hollow and includes a central passageway 26 for the controlled passage of food, fluids and medications therethrough in a manner known to those skilled in the art. The retention shaft 12 further includes a one way valve 70 positioned within the central passageway 26 to prevent return of infused materials delivered via the gastrostomy tube 1 and gastric content in a manner known to those skilled in the art. The elongated hollow retention shaft 12 includes a first end 22 and a second end 23. At one end, that is, the first end 22, of the hollow retention shaft 12 a retention shaft collar 14 is positioned. The retention shaft collar 14 includes an opening 15 in communication with the interior hollow central passageway 26 of the retention shaft 12. The hollow central passageway 26 runs longitudinally within the retention shaft 12 and runs the full length of the retention shaft 12 from the proximal first end 22 of the retention shaft 12 at the retention shaft collar 14 to the opposite distal second end 23 of the retention shaft 12.

As briefly discussed above, the retention device 10 also includes a concentric outer sleeve 13 that fits about the retention shaft 12 for movement relative thereto. The concentric outer sleeve 13 includes a first end 27 and a second end 39. The first end 27 is provided with a sleeve collar 71 shaped and dimensioned for engagement with the retention shaft collar 14.

The sleeve collar 71 includes an upwardly facing surface 72 that faces the retention shaft collar 14. The upwardly facing surface 72 includes a central annular recess 73 following the outer edge of the circumference defined by the central passageway 29 of the concentric outer sleeve 13. The annular recess 73 is positioned such that the retention shaft collar 14 is seated therein when the retention device 10 is in the open configuration as shown in FIG. 1B. The ability of the retention shaft collar 14 to seat within the recess 73 formed about the sleeve collar 71 enhances the profile of the gastrostomy tube 1 by limiting the upward extent thereof by at least the thickness of the retention shaft collar 14 that would otherwise have to sit above the sleeve collar 71.

As will be discussed below in greater detail, the sleeve collar 71 at the first end 27 of the concentric outer sleeve 13 is provided with a button 16 for closing the opening 15. The button 16 is shaped and dimensioned for selectively closing the opening 15 formed in the first end 22 of the retention shaft 12. The button 16 is attached to the first end 27 of the concentric outer sleeve 13 by a tether 17. At a distal end 28 of the tether 17 on a side of the button 16 opposite that of the first end 27 of the concentric outer sleeve 13 and the opening 15, the tether 17 is provided with a tab 18 for handling the button 16 for closing the opening 15. Although not disclosed in accordance with this embodiment of the invention, U.S. Provisional Patent Application Ser. No. 61/356,694, entitled “RETENTION DEVICE FOR GASTROSTOMY TUBE”, filed Jun. 21, 2010, which is incorporated herein by reference, shows that it is appreciated the button can be formed in the first end of the retention shaft. The button is attached to the collar by a tether. At a distal end of the tether on a side of the button opposite that of the collar and the opening can be a tab for handling the button for closing and opening.

At the distal second end 23 of the retention shaft 12, opposite the first end 22 where the retention shaft collar 14 is formed, are one or more retention members 20 for securing the retention device 10 in place. In the embodiment illustrated in FIGS. 1A and 1B, the retention members 20 consist of a plurality of fingers or splines 20. In one embodiment, the splines 20 are normally resiliently biased in an expanded or splayed configuration, where they extend in a direction substantially transverse to the longitudinal axis of the retention shaft 12, such as illustrated in FIG. 1B. However, and in accordance with an alternate embodiment as explained below, it is appreciated the retention device may be constructed with a mechanism for selectively extending or retracting the splines 20.

As briefly discussed above, the retention device 10 includes a retention shaft assembly 11 and a concentric outer sleeve 13 that wraps about the retention shaft assembly 11, in particular, the retention shaft 12. The concentric outer sleeve 13 is positioned about the retention shaft 12 such that is free to slide longitudinally along the outside of the retention shaft 12. As such, the inner diameter of the concentric outer sleeve 13 is slightly larger than the outer diameter of the retention shaft 12.

A locking mechanism 25 is provided for selectively locking the retention shaft 12 relative to the concentric outer sleeve 13. The locking mechanism 25 permits the concentric outer sleeve 13 to engage the retention shaft 12 such that the concentric outer sleeve 13 may be locked in any one of a number of positions or locations along the retention shaft 12. The locking mechanism 25 is composed of outwardly extending knobs 30 formed on opposite sides of the retention shaft 12 adjacent the first end 22 of the retention shaft 12, the knobs 30 are shaped and dimensioned for positioning within holes (or recesses) 31 formed along the inner wall 32 of the concentric outer sleeve 13 on opposite sides thereof. The knobs 30 are formed of a soft, pliable material allowing the retention shaft 12 in the area of the knobs 30 to inwardly extend for positioning within the concentric outer sleeve 13 when the retention shaft 12 is slid downwardly within the concentric outer sleeve 13 and extends outwardly when the knobs come into alignment with the holes 31 formed in the inner wall 32 of the concentric outer sleeve 13 such that the knobs 30 seat therein to lock the retention shaft 12 relative to the concentric outer sleeve 13. Alignment of the retention shaft 12 with the concentric outer sleeve 13 in a manner allowing for controlled engagement of the knobs 30 with the holes 31 is achieved through the provision of guide slots 33 formed in the inner wall 32 between the upper edge 61 of the concentric outer sleeve 13 at the first end 27 of the outer sleeve 13 and the holes 31 formed in the inner wall 32 of the concentric outer sleeve 13. It is appreciated multiple longitudinally spaced holes may be provided along the inner wall of the sleeve such that the retention shaft may be locked at various positions relative to the sleeve.

When it is time to disengage the retention shaft 12 from the concentric outer sleeve 13 for removal of the retention device 10, a removal device 34 (see FIG. 1C) is slid down within the guide slots 33 (or notch) formed along the inner wall 32 of the concentric outer sleeve 13 until it engages the knobs 30 with sufficient force to disengage the knobs 30 from their position within the holes 31 so that the retention shaft 12 may be pulled upwardly within the concentric outer sleeve 13. In particular, the removal device 34 is formed with removal shafts 35 having laterally extending protrusions 36 at their respective distal ends 37. The laterally extending protrusions 36 allow the removal shafts 35 to be pushed past the knobs 30 as the shafts 35 force the knobs 30 from their positions within the holes 31, such that the laterally extending protrusions 36 engage the respective bottom surfaces 67 of the knobs 30 in a manner allowing one to pull upwardly on the removal device 34 in a manner pulling the retention shaft 12 upwardly until the retention members 20 are drawn inwardly to their retracted position.

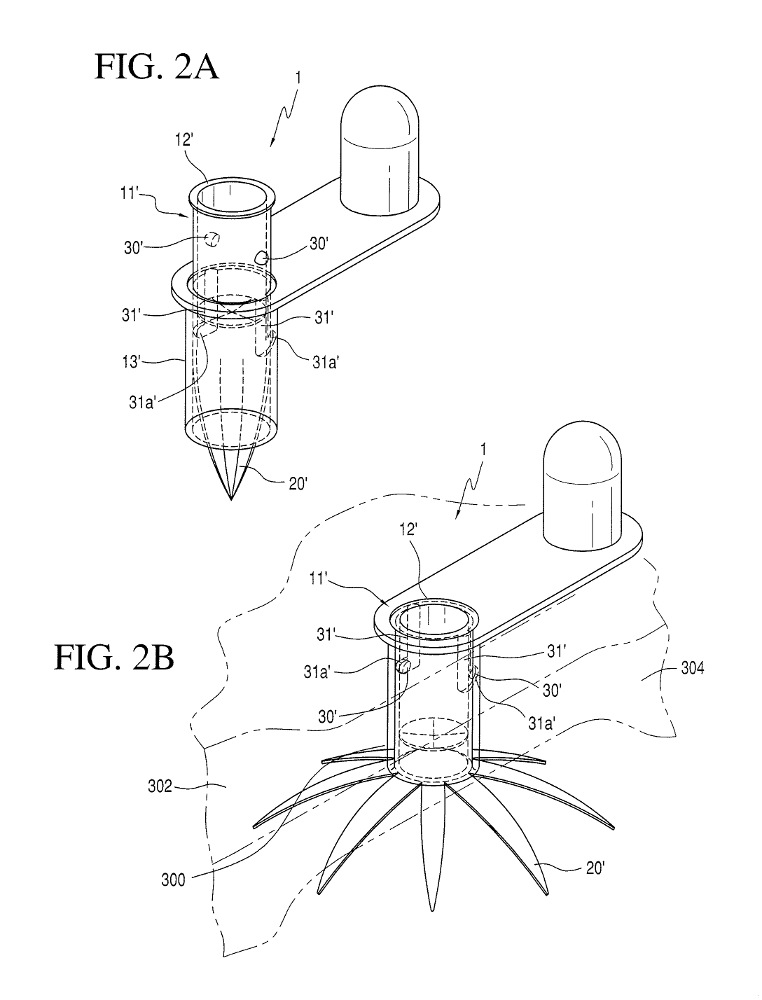

It is appreciated other locking mechanism may be employed for controlling the positioning of the retention shaft assembly relative to the concentric outer sleeve. For example, and with reference to FIGS. 2A and 2B, a bayonet locking mechanism 25′ is contemplated. In accordance with such an embodiment, the retention shaft 12′ is provided with outwardly extending protrusions 30′ shaped and dimensioned for receipt within guiding slots 31′ formed along the inner wall of the concentric outer sleeve 13′. Once the retention shaft assembly 11′ is pushed downwardly relative to the concentric outer sleeve 13′ and the splines 20′ expand outwardly, the guiding slots 31′, which are L-shaped, permit rotation of the retention shaft assembly 11′ causing the protrusions 30′ to move within the lateral portions 31a′ of the guide slots 31′ to effectively lock the retention shaft assembly 11′ relative to the concentric outer sleeve 13′ by preventing longitudinal movement relative thereto.

In practice, and with reference to FIGS. 1A and 1B, when the concentric outer sleeve 13 is moved along the retention shaft 12 such that sleeve collar 71 moves away from the retention shaft collar 14, as illustrated in FIG. 1A, that is, when the retention shaft 12 is drawn upwardly such that the retention shaft collar 14 at the first end 22 of the retention shaft 12 moves away from the sleeve collar 71 at the first end 27 of the concentric outer sleeve 13 and the second end 23 of the retention shaft 12 is drawn within the concentric outer sleeve 13, the retention members 20 move to a collapsed or retracted position appropriate for deployment within a passageway formed in the body. In particular, when the second end 23 of the retention shaft 12 is drawn within the concentric outer sleeve 13, the retention members 20 contact the lower edge 38 of the concentric outer sleeve 13 at the second end 39 of the concentric outer sleeve 13 in a manner causing the retention members 20 to collapse together in an elongated configuration (that is, the longitudinal axes of the splines 20 are substantially parallel with the longitudinal axis of the retention shaft 12) allowing the retention device 10 to be inserted through a body passageway 300 formed through the skin 302 and stomach wall 304 or removed from the body passageway 300 formed through the skin 302 and stomach wall 304 (see FIGS. 1B and 2B). In this configuration, the distal end 60 of the retention members 20 can be formed such that they collapse upon each other forming a dull point allowing for ease of insertion through the passageway formed in the body.

Once the gastrostomy tube 1 is inserted through the passageway formed in the body with the distal end 60 of the retention members 20 positioned within the stomach, the retention device 10 of the gastrostomy tube 1 may be actuated to move to its open orientation shown in FIG. 1B. In this open orientation, the retention device 10 locks the gastrostomy tube 1 in position with the skin 302 and stomach wall 304 adjacent the body passageway 300 held between the outwardly extending spines 20 and the sleeve collar 71.

With the gastrostomy tube 1 positioned within the body passageway 300 and the sleeve collar 71 in direct contact with the exterior surface of the passageway 300 at the skin 302 surface, slight downwardly pressure, that is pressure pushing the retention shaft collar 14 toward the sleeve collar 71, may be applied to overcome the frictional resistance holding the retention shaft assembly 11 relative to the concentric outer sleeve 13. It is appreciated the frictional resistance is primarily a function of the interaction between the outwardly biased splines 20 and the second end 23 of the concentric outer sleeve 13. The retention shaft 12 is then slid along the concentric outer sleeve 13 moving the retention shaft collar 14 toward the sleeve collar 71 and the retention members 20 away from the second end 39 of the concentric outer sleeve 13 thus exposing the retention members 20 allowing the retention members 20 to splay outwardly. Movement of the retention shaft 12 relative to the concentric outer sleeve 13 continues until the retention shaft collar 14 seats within the annular recess 73 formed along the sleeve collar 71.

With the concentric outer sleeve 13 and the retention shaft 12 free to move relative to each other, the retention shaft collar 14 at the first end 22 of the retention shaft 12 is moved toward the sleeve collar 71 at the first end 27 of the concentric outer sleeve 13 causing the retention members 20 to be moved away from the second end 39 of the concentric outer sleeve 13. Once the proximal ends 62 of the retention members 20 are moved from contact with the second end 39 of the concentric outer sleeve 13, their inherent outward bias causes them to move outwardly (such that the longitudinal axes of the splines 20 are substantially transverse to the longitudinal axis of the retention shaft 12) into an open orientation as shown in FIG. 1B.

Once the retention shaft collar 14 is fully seated within the annular recess 73 of the sleeve collar 71, the locking mechanism 25 should be fully engaged. That is, the knobs 30 have been moved within the guide slots 33 formed along the inner wall 32 of the concentric outer sleeve 13 to their locked positions within the holes 31. With the knobs 30 positioned within the holes 31, the locking mechanism 25 securely holds the concentric outer sleeve 13 and the retention shaft assembly 11 in this opened orientation (see FIG. 1B), securing the retention device 10, and ultimately, the gastrostomy tube 1, in place by positioning the skin 302 and stomach wall 304 adjacent the body passageway 300 between the outwardly extending splines 20 and the sleeve collar 71.

To remove the retention device 10, the locking mechanism 25 may be engaged by the removal device 34, and the retention shaft assembly 11 is moved longitudinally along the concentric outer sleeve 13 such that the retention shaft collar 14 and the sleeve collar 71 are moved apart causing the retention members 20 to collapse and allowing the retention device 10, and ultimately, the gastrostomy tube 1, to be withdrawn from the body passageway 300. That is, when it is time to disengage the retention shaft assembly 11 from the concentric outer sleeve 13 for removal of the gastrostomy tube 1, the removal device 34 is slid down within guide slots 33 formed along the inner wall 32 of the concentric outer sleeve 13 until it engages the knobs 30 with sufficient force to disengage the knobs 30 from their positions within the holes 31 so that the retention shaft assembly 11 may be pulled upwardly within the concentric outer sleeve 13. The lateral extending protrusions 36 are pushed past the knobs 30 as they force the knobs 30 from their positions within the holes 31, such that the laterally extending protrusions 36 engage the bottom surface 67 of the knobs 30 in a manner allowing one to pull upwardly on the removal device 34 in a manner pulling the retention shaft 12 upwardly until the retention members 20 are drawn inwardly to their retracted position. When the second end 23 of the retention shaft 12 is drawn within the concentric outer sleeve 13, the proximal end 62 of the retention members 20 contact the lower edge 38 of the concentric outer sleeve 13 at the second end 39 of the concentric outer sleeve 13 in a manner causing the retention members 20 to collapse together in an elongated configuration.

It is appreciated that if a bayonet type locking mechanism were employed, the disengagement would involve the rotation of the retention shaft relative to the concentric outer sleeve.

In one exemplary form, the fingers or splines 20 are connected to the distal second end 23 of the retention shaft 12 and are formed of a material, such as a shape memory alloy, that normally biases the splines 20 outwardly in splayed fashion to the open orientation shown in FIG. 1B or 2B. In another embodiment, it is appreciated the splines may be formed having a hinged mechanism analogous to an umbrella allowing the fingers or splines to flex outwardly or alternatively be retracted and collapsed together.

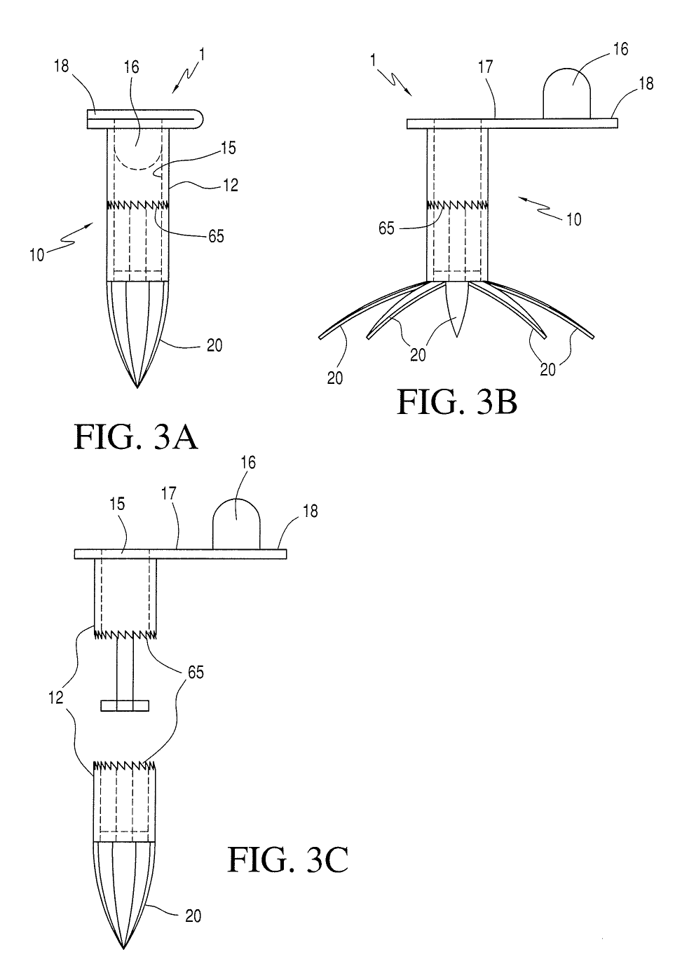

In yet another embodiment, as illustrated in FIGS. 3A-3C, the mechanism for the concentric outer sleeve 13 for releasing or retracting, that is, moving the retention device between the open orientation and the retracted orientation, the splines 20 as disclosed above with reference to the embodiment disclosed in FIGS. 1A-1C, may be replaced with an alternative mechanism for releasing or retracting the splines. FIGS. 3A-3C illustrate, for example, a saw tooth lock mechanism 65 that may be used in controlling the extension of the splines to an open orientation and retraction of the splines 20 to the retracted orientation. In this saw tooth lock mechanism 65, the retention shaft 12 is twisted in one direction or the other to extend or collapse (that is, retract) the splines 20. FIG. 3C illustrates an exploded view of the mechanism.

In another embodiment (not shown), the retention shaft may incorporate a threaded collar and mating threaded sleeve. Retention members may be coupled to the threaded collar. Rotation of the threaded sleeve about the retention shaft will cause the threaded collar to travel towards or away from the collar causing the retention members to selectively extend and splay outwardly for retaining the retention device in place or alternatively be withdrawn causing the retention members to collapse upon themselves allowing for the retention device to be withdrawn from the body passageway.

In another exemplary embodiment, the retention device of the present disclosure includes a mesh member at one end of the retention member, which mesh member is normally biased to an expanded form, such as an ovoid. An introducer may be inserted through the hollow shaft of the retention member and used to extend and straighten out the mesh device to an elongated condition allowing the retention member to be inserted through the body passageway. The introducer can then be removed allowing the mesh member to spring back to its normally biased expanded configuration to retain the device in place. To remove the retention member, the introducer may be reinserted through the hollow shaft of the retention member to extend the mesh to an elongated condition allowing for removal of the retention device from the body passageway.

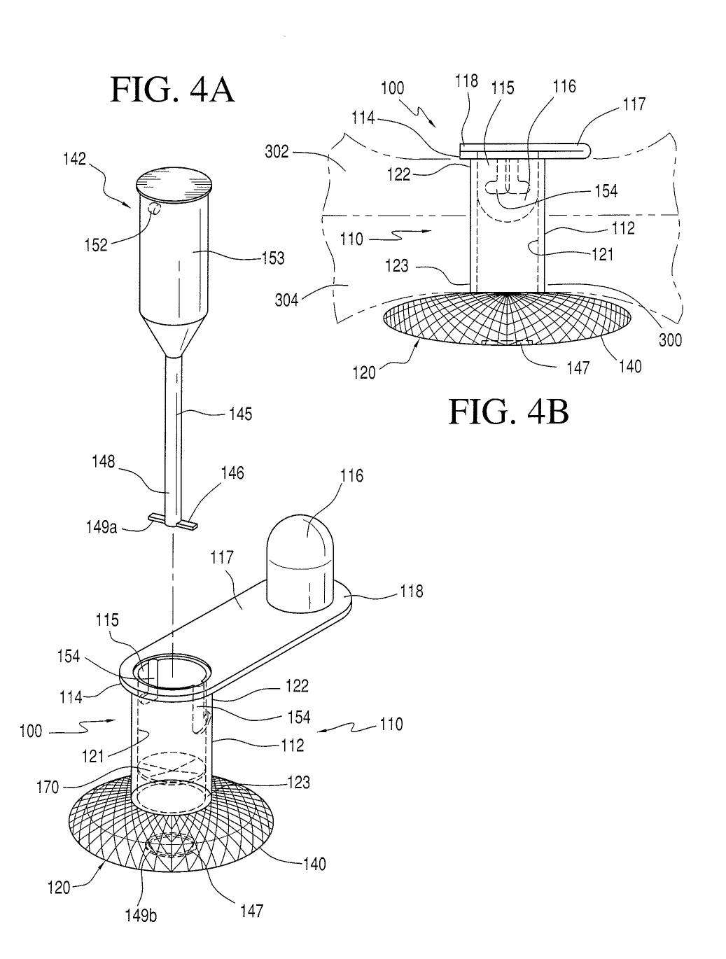

More particularly, this embodiment is illustrated in FIGS. 4A-4C. The retention device 110 of the gastrostomy tube 100 incorporates a mesh-like device 140. The retention device 110 includes a hollow retention shaft 112 with a retention member 120 in the form of a mesh-like device 140 extending therefrom. In accordance with a preferred embodiment, the mesh device 140 is composed of interlocking strands of shape memory metal alloy material adapted to take a disk shape upon deployment. The retention shaft 112 is hollow and includes a central passageway 121 through which the food may be passed. The central passageway 121 is also provided with a one-way valve 170 as discussed above with regard to the embodiment disclosed in FIG. 1A-C. The elongated hollow retention shaft 112 includes a first end 122 and a second end 123. At one end, that is, the first end 122, of the hollow retention shaft 112 a retention shaft collar 114 is positioned. The retention shaft collar 114 includes an opening 115 in communication with an interior hollow central passageway 121 of the retention shaft 112. The hollow central passageway 121 runs longitudinally within the retention shaft 112 and runs the full length of the retention shaft 112 from the proximal first end 122 of the retention shaft 112 at the retention shaft collar 114 to its opposite distal second end 123 of the retention shaft 112.

The first end 122 of the retention shaft 112 adjacent the retention shaft collar 114 is provided with a button 116 for closing the opening 115. The button 116 is shaped and dimensioned for selectively closing the opening 115 formed in the first end 122 of the retention shaft 112. The button 116 is attached to the retention shaft collar 114 at the first end 122 of the retention shaft 112 by a tether 117. At a distal end of the tether 117 on a side of the button 116 opposite that of the retention shaft collar 114 and the opening 115 can be a tab 118 for handling the button 116 for closing the opening 115.

The mesh device 140 may be, for example, of a metal such as shape memory metal alloy (as discussed above), or a plastic material, similar to a cardiac stent. The mesh device 140 is designed such that the mesh device 140 is normally biased in an outwardly expanded, open orientation such as illustrated in FIG. 4A. To insert the retention device 110 of this embodiment through a body passageway 300 in the skin 302 and stomach wall 304 and insert the mesh device 140 into the stomach, an introducer 142 is used to elongate the mesh device 140 and thereby reduce its diameter to a retracted orientation (that is, a low profile extended, orientation). The introducer 142 includes a long shaft 145 having tabs (or a collar) 146 at one end. The shaft 145 of the introducer 142 is inserted through the opening 115 of the retention shaft 112 of the retention device 110 into the hollow central passageway 121 of the retention shaft 112 of the retention device 110 and through the central passageway 121 defined by the retention shaft 112. The tabs 146 at the distal end of the shaft 145 are then pushed against the mesh device 140 at the distal end 144 of the mesh device 140 causing the mesh device 140 to extend into an elongated configuration, that is, its retracted orientation, collapsing upon itself for introduction into the body passageway 300 formed through the skin 302 and stomach wall 304.

Engagement of the shaft 145 with the distal end 144 of the mesh device 140 is facilitated by the provision of an opening ring 147 at the distal end 144 of the mesh device 140. The opening ring 147 is shaped and dimensioned for engagement with the distal end 148 of the shaft 145. In particular, the distal end 148 of the shaft 145 and the opening ring 147 are provided with mating ledges 149a, 149b such that the distal end 148 of the shaft 145 seats along the opening ring 147.

Proper positioning of the introducer 142 relative to the retention device 110, and controlled retention of the mesh device 140 in its low profile extended configuration with the shaft 145 of the introducer 142 fully extended therein, is facilitated by the provision of an interlocking mechanism associated with the retention shaft 112 and the proximal end 143 of the shaft 145 of the introducer 142. In particular, lugs or nibs 152 are formed on the outer surface 153 of the proximal end 143 of the shaft 145 of the introducer 142 on opposite thereof and one or more associated notched paths 154 are formed along the interior wall of the hollow passageway 121 of the retention shaft 112 (preferably two formed on opposite sides of the retention shaft). The nibs 152 and the notched paths 154 are shaped and dimensioned for selectively connecting the introducer 142 to the gastrostomy tube 100 including the retention device 110.

To connect the introducer 142 to the retention device 110, the distal end 148 of the introducer 142 is inserted into the opening 115 of the retention shaft 112 and into the hollow passageway 121 of the retention shaft 112. The distal end of the shaft 145 is then seated upon the opening ring 147. In this position, the lugs or nibs 152 engage the notched path 154 in the interior hollow passageway 121 of the retention shaft 112, and the introducer 142 is then rotated causing the lugs or nibs 152 to lock into place within the notched path 154. The introducer 142 may be disengaged from the retention shaft 112 by simply rotating the shaft 145 in the opposite direction.

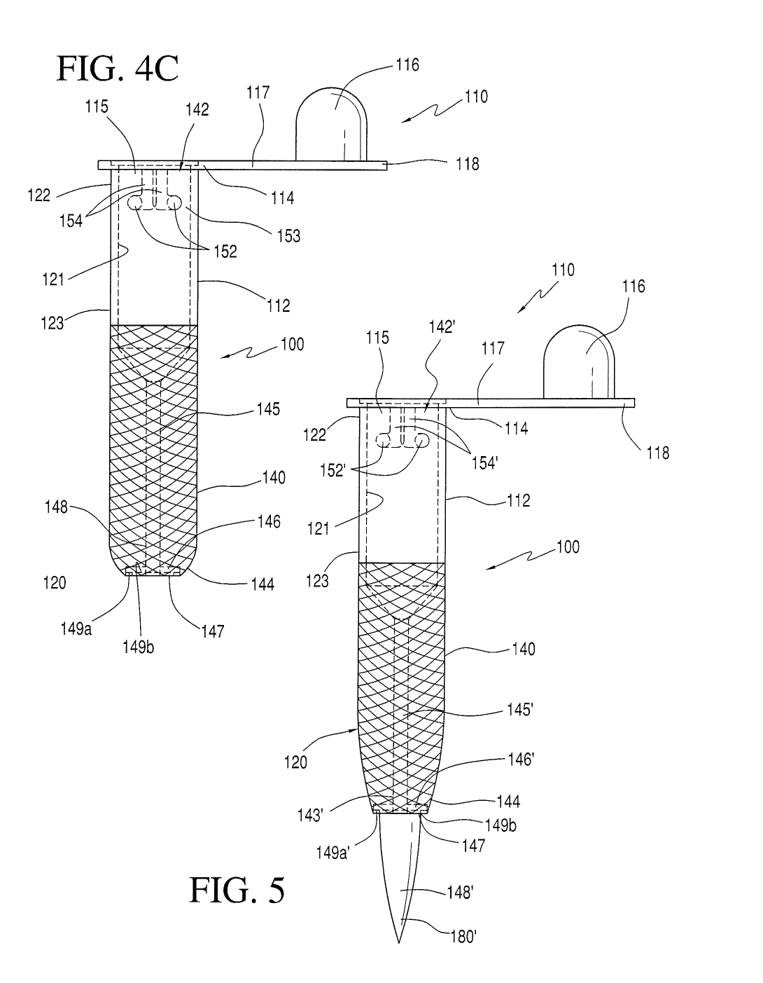

Once the retention shaft 112 of the retention device 110 is inserted and in place, the introducer 142 may be removed allowing the mesh device 140 to expand to its normal expanded biased condition, that is, its open position, causing the retention device 110 to be held in place with the skin 302 and stomach wall 304 adjacent the body passageway 300 held between the mesh device 140 in its open orientation and the retention shaft collar 114 at the first end 122 of the retention shaft 112. Passage of food through both the mesh device 140 and the retention shaft 112 is achieved through the provision of an opening ring 147 in the mesh device 140. The opening ring 147 is aligned with the central passageway 121 of the retention shaft 112 to permit passage of the gastrostomy tube through both the retention shaft 112 and the mesh device 140.

Where a stoma is not present prior insertion of the gastrostomy tube 100, the retention device 112 may be deployed using an alternate introducer 142′ formed with a pointed tip 180′ similar to those found in conjunction with obturators. Such an embodiment is disclosed with reference to FIG. 5. As with the prior embodiment, introducer 142′ includes a long shaft 145′ having tabs (or a collar) 146′ at one end, that is, the proximal end 143′. In contrast to the prior embodiment, however, the distal end 148′ of the shaft 145′ is provided with a pointed tip 180′ shaped and dimensioned for the creation of a hole, simultaneously with the deployment of the gastrostomy tube 100, in tissue as forward force is applied.

The pointed tip 180′ is shaped and dimensioned for positioning within the opening ring 147 at the end of the mesh device 140, such that the pointed tip 180′ passes through the opening ring 147 for engagement with tissue as the medical practitioner applies pressure in the creation of a passageway. As with previously described introducer 142, alignment is further achieved by providing the second end of the shaft 145′, proximal to the pointed tip 180′, with a ledge 149a shaped and dimensioned to mate with a similar ledge surface 149b formed along the opening ring 147.

With the introducer 142′ secured to the retention device 112 the assembly is positioned at a desired location and downward pressure is applied causing the pointed tip 180′ to pass through the tissue forming a stoma. Once the gastrostomy tube 100 is properly positioned, the introducer 142′ is rotated allowing the nib 152′ on the introducer 142′ to disengage from notch path 154′ ‘on the retention shaft 112 and the introducer 142′ is removed allowing the mesh device 140 to expand to its normal expanded biased condition taking its open orientation causing the retention device 110 to be held in place with the skin and tissue held between the retention shaft collar 114′ and the mesh device 140.

To remove the retention device 110 of this embodiment, the introducer 142′ may be extended into and through the hollow central passageway 121 of the retention shaft 112 causing the mesh device 140 to extend longitudinally and collapse upon itself allowing the retention device 110 to be removed from the body passageway.

It should also be emphasized that the above-described embodiments of the present disclosure are merely possible examples of implementations set forth for a clear understanding of the principles of the present disclosure. Many variations and modifications may be made to the above-described embodiments without departing substantially from the spirit and principles of the disclosure. All such modifications and variations are intended to be included herein within the scope of this disclosure and protected by the following claims.

While the preferred embodiments have been shown and described, it will be understood that there is no intent to limit the invention by such disclosure, but rather, is intended to cover all modifications and alternate constructions falling within the spirit and scope of the invention.

1. A gastrostomy tube including a retention device, comprising:
an elongated hollow shaft having a proximal first end and a distal second end;
at least one retention member extending from the distal second end of the shaft;
wherein the at least one retention member moves between a retracted orientation and an open orientation.

2. The gastrostomy tube according to claim 1, wherein the at least one retention member is a spline resiliently biased in a splayed configuration.

3. The gastrostomy tube according to claim 2, wherein the spline is composed of a shape memory alloy.

4. The gastrostomy tube according to claim 2, wherein the at least one retention member includes a plurality of splines resiliently biased in a splayed configuration.

5. The gastrostomy tube according to claim 1, further including a concentric sleeve wrapped about the shaft for sliding longitudinally along the shaft.

6. The gastrostomy tube according to claim 5, wherein the at least one retention member is a spline resiliently biased in a splayed configuration.

7. The gastrostomy tube according to claim 6, wherein movement of the sleeve relative to the shaft causes the at least one retention member to move between the retracted orientation and the open orientation.

8. The gastrostomy tube according to claim 7, wherein a lower edge of the sleeve contacts a proximal end of the at least one retention member causing the at least one retention member to move to the retracted orientation.

9. The gastrostomy tube according to claim 8, wherein the spline is composed of a shape memory alloy.

10. The gastrostomy tube according to claim 5, further including a locking mechanism for selectively locking the shaft relative to the sleeve.

11. The gastrostomy tube according to claim 1, further including a button for closing the hollow shaft.

12. The gastrostomy tube according to claim 1, wherein the at least one retention member is a mesh device biased to an expanded form.

13. The gastrostomy tube according to claim 12, wherein the mesh device is a shape memory metal alloy

14. The gastrostomy tube according to claim 12, wherein the mesh device is a plastic material.

15. The gastrostomy tube according to claim 12, further including an introducer used in controlling the orientation of the mesh device.

16. The gastrostomy tube according to claim 15, wherein the introducer includes a shaft having a distal end shaped and dimensioned to engage the mesh device.

17. The gastrostomy tube according to claim 15, wherein the distal end of the shaft includes a point tip.
